(19) United States
(12) Patent Application Publication
(10) Pub. No.: US 2023/0368894 A1
(43) Pub. Date: Nov. 16, 2023

(54) SYSTEMS AND METHODS FOR PROCESSING ELECTRONIC IMAGES WITH UPDATED PROTOCOLS

(71) Applicant: PAIGE.AI, Inc., New York, NY (US)

(72) Inventors: Jeremy Daniel KUNZ, New York, NY (US); Donghun LEE, New York, NY (US)

(21) Appl. No.: 18/316,736

(22) Filed: May 12, 2023

Related U.S. Application Data
(60) Provisional application No. 63/341,825, filed on May 13, 2022.

(51) Int. Cl. G16H 30/20; G06T 7/00; G06V 10/74

(52) U.S. Cl. CPC G16H 30/20; G06T 7/0012; G06V 10/761; G06T 2207/20081

(57) ABSTRACT

Systems and methods are described herein for processing electronic medical images. For example, one or more digital medical images of at least one pathology specimen, the pathology specimen being associated with a patient may be received. Additionally, an external designation of the one or more digital medical images may be received. The one or more digital medical images may be provided to one or more machine learning systems, the one or more machine learning systems each having been trained to analyze medical images using one of a plurality of versions of a protocol. The one or more machine learning systems, may determine machine learning system designations for the one or more digital medical images. The external designation may be compared to the machine learning system designations and, based on the comparison, determining whether the external designation matches a predetermined protocol.

### CROSS-REFERENCE TO RELATED APPLICATION(S)

This application claims the benefit of priority to U.S. Provisional Patent Application No. 63/341,825, filed May 13, 2022, the entirety of which is incorporated by reference. Further, U.S. Provisional Patent Application No. 63/341,504, filed May 13, 2022 is incorporated by reference in its entirety.

### TECHNICAL FIELD

Various embodiments of the present disclosure pertain generally to image processing. More specifically, particular embodiments of the present disclosure relate to systems and methods to updating protocols for processing electronic images.

### BACKGROUND

Within the field of pathology, hospitals and the College of American Pathologists (CAP) establish guidelines, existing protocols, synoptic reports, and worksheets (collectively referred to as “guidelines” or “protocols”) for pathologist and researchers to adhere to during analysis. The guidelines may provide standard practices and reporting techniques for pathologist/researchers. These guidelines may be updated frequently and pathologist and hospital workers may need to stay up to date with the best practices based on the guidelines.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the present disclosure, systems and methods are disclosed for processing electronic medical images. For example, one or more digital medical images of at least one pathology specimen, the pathology specimen being associated with a patient may be received. Additionally, an external designation of the one or more digital medical images may be received. The one or more digital medical images may be provided to one or more machine learning systems, the one or more machine learning systems each having been trained to analyze medical images using one of a plurality of versions of a protocol. The one or more machine learning systems, may determine machine learning system designations for the one or more digital medical images. The external designation may be compared to the machine learning system designations and, based on the comparison, determining whether the external designation matches a predetermined protocol.

The predetermined protocol may correspond to a most recently determined protocol. The method may further comprise outputting to one or more users that the predetermined protocol was used in the external designation and outputting whether the external designation does not match any of the machine learning system designations.

The method may further comprise, upon determining the external designation does not match the predetermined protocol, outputting the predetermined protocol to a user or external system and outputting the protocol of the machine learning designation that most closely matches the external designation.

The method may further comprise determining a new protocol has been developed; updating the predetermined protocol to be the new protocol; and determining training a new machine learning system based on the new protocol.

According to certain aspects of the present disclosure, systems and methods are disclosed for processing electronic medical images. In another aspect, a system for processing electronic digital medical images may comprise at least one memory storing instructions and at least one processor configured to execute the instructions to perform operations. The at least one processor may comprise processing electronic medical images. For example, one or more digital medical images of at least one pathology specimen, the pathology specimen being associated with a patient may be received. Additionally, an external designation of the one or more digital medical images may be received. The one or more digital medical images may be provided to one or more machine learning systems, the one or more machine learning systems each having been trained to analyze medical images using one of a plurality of versions of a protocol. The one or more machine learning systems, may determine machine learning system designations for the one or more digital medical images. The external designation may be compared to the machine learning system designations and, based on the comparison, determining whether the external designation matches a predetermined protocol.

The predetermined protocol may correspond to a most recently determined protocol. The system may further comprise outputting to one or more users that the predetermined protocol was used in the external designation and outputting whether the external designation does not match any of the machine learning system designations.

The system may further comprise, upon determining the external designation does not match the predetermined protocol, outputting the predetermined protocol to a user or external system and outputting the protocol of the machine learning designation that most closely matches the external designation.

The system may further comprise determining a new protocol has been developed; updating the predetermined protocol to be the new protocol; and determining training a new machine learning system based on the new protocol.

According to certain aspects of the present disclosure, systems and methods are disclosed for processing electronic medical images. In another aspect, a non-transitory computer-readable medium storing instructions that, when executed by a processor, perform operations processing electronic digital medical images, is disclosed. The operations may include receiving one or more digital medical images of at least one pathology specimen, the pathology specimen being associated with a patient. Additionally, an external designation of the one or more digital medical images may be received. The one or more digital medical images may be provided to one or more machine learning systems, the one or more machine learning systems each having been trained to analyze medical images using one of a plurality of versions of a protocol. The one or more machine learning systems, may determine machine learning system designations for the one or more digital medical images. The external designation may be compared to the machine learning system designations and, based on the comparison, determining whether the external designation matches a predetermined protocol.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims. As will be apparent from the embodiments below, an advantage to the disclosed systems and methods is that multiple parties may fully utilize their data without allowing others to have direct access to raw data. The disclosed systems and methods discussed below may allow advertisers to understand users' online behaviors through the indirect use of raw data and may maintain privacy of the users and the data.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1A illustrates an exemplary block diagram of a system and network for processing images to determine an external designation protocol, according to techniques presented herein.

FIG. 1B illustrates an exemplary block diagram of a tissue viewing platform according to techniques presented herein.

FIG. 1C illustrates an exemplary block diagram of the slide analysis tool 101, according to an exemplary embodiment of the present disclosure.

FIG. 2 depicts an exemplary section of a College of American Pathologist (CAP) protocol.

FIG. 3 depicts an exemplary block diagram of a system and network to determine an external designation's protocol, according to an exemplary embodiment of the present disclosure.

FIG. 4 is a flowchart of an example method for training a system to determine an external designation's protocol, according to techniques presented herein.

FIG. 5 is a flowchart illustrating an example method for using a trained system to determine an external designation's protocol, according to one or more exemplary embodiments herein.

FIG. 6 is a flowchart illustrating an example method for determining an external designation's protocol, according to one or more exemplary embodiments herein.

FIG. 7 depicts an example of a computing device that may execute techniques presented herein, according to one or more embodiments.

### DETAILED DESCRIPTION OF EMBODIMENTS

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

As used herein, a “machine learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine learning model is generally trained using training data, e.g., experiential data and/or samples of input data, which are fed into the model in order to establish, tune, or modify one or more aspects of the model, e.g., the weights, biases, criteria for forming classifications or clusters, or the like. Deep learning techniques may also be employed. Aspects of a machine learning model may operate on an input linearly, in parallel, via a network (e.g., a neural network), or via any suitable configuration.

In the field of pathology, a pathologist may be required to adhere to specific guidelines/protocols (e.g., CAP guidelines, existing protocol, synoptic reports, and/or worksheets) when performing analysis. Adhering to guidelines may help pathologist and laboratory professional to provide more effective testing with consistent, high-quality results, and expert interpretations. Exemplary guidelines may provide information for collecting the essential data elements for complete reporting of malignant tumors an optimal patient care. Guidelines may be updated at particular times, as standards evolve within a field. Thus, particular guidelines may include multiple versions as the documents are updated. Pathologist and hospitals may be under pressures to stay up to date and utilize the preferred versions of these guidelines. Pathologist may have their measurements and/or diagnosis reviewed to make sure appropriate versions of a guideline are utilized using techniques discussed herein. In addition, techniques disclosed herein may ensure that the correct protocol is outputted to a pathologist utilizing an older version of a guideline.

Pathology departments and/or laboratories are also under pressure to make sure that pathologist are utilizing the most up to date guidelines at their institutions. A system that analyzes pathologist's measurements and diagnosis to verify that correct guidelines are being utilized may be advantageous to labs and their clients.

In one example, the system and methods described herein may first receive one or more digital medical images. Next, a pathologist may determine a diagnosis (or diagnoses) and/or measurement(s) on the inputted digital medical images according to a particular protocol. The system may then receive the inputted diagnosis and/or measurement as an external designation. An external designation may refer to metadata of a measurement and/or a diagnosis, and/or annotations of a digital medical image. Further, an external designation may refer to any field a medical expert fills out in a standardized document, a guideline, medical report, and/or a protocol. The system may then determine machine learning systems for each version of the protocol applied within the external designation. The system may then apply the determined machine learning systems to the inputted digital medical images and output a measurement and/or diagnosis. Next, the system may compare the external designation of the pathologist with the diagnosis and/or measurements of the machine learning systems. The system may then determine which version of the guidelines was utilized by the pathologist (e.g., perform a similarity match). If the pathologist used the latest version of the guidelines, the system may end analysis of the digital medical images and record that the pathologist used the latest guidelines. If the system matches the external designation with a previous version of the guidelines, the system may notify the pathologist that they are using an outdated guideline and further provide the latest guidelines to the user.

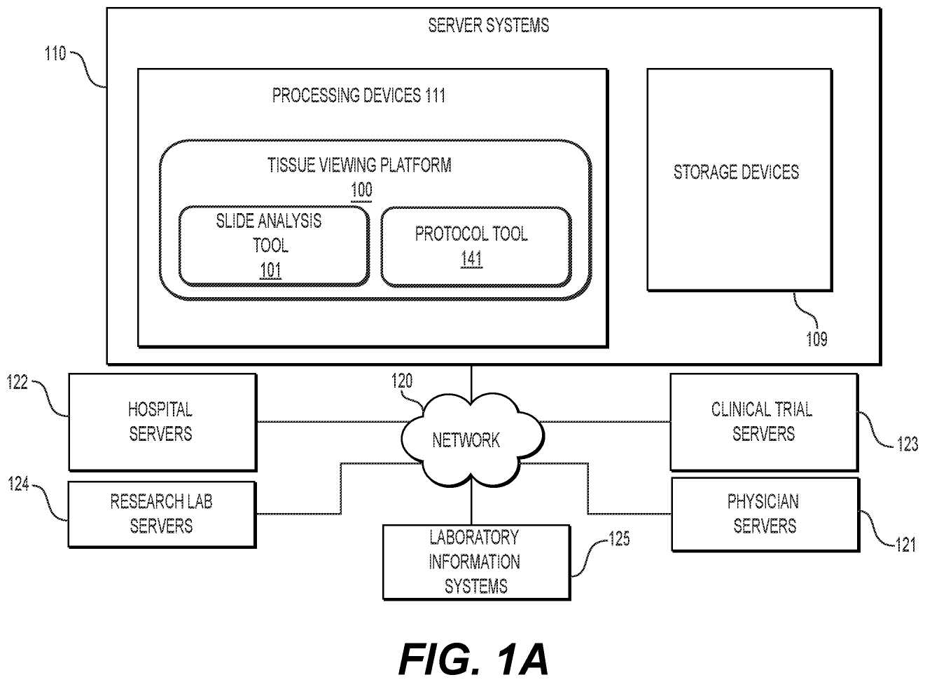

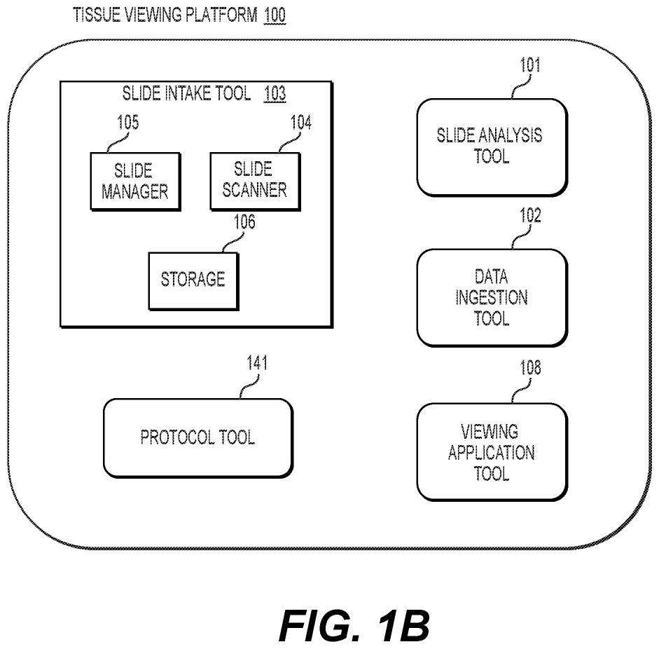

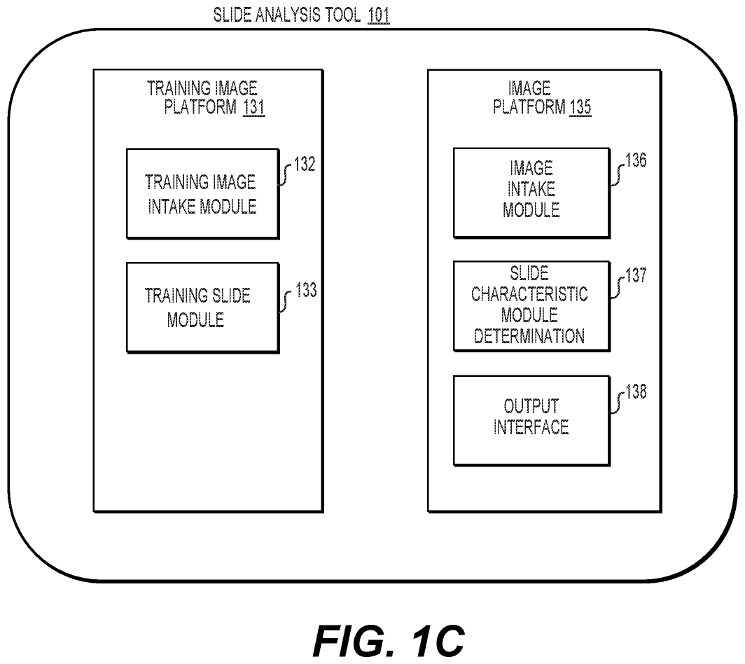

FIG. 1A illustrates a block diagram of a system and network for processing images to determine an external designation protocol, according to an exemplary technique of the present disclosure.

Specifically, FIG. 1A illustrates an electronic network 120 that may be connected to servers at hospitals, laboratories, and/or doctors' offices, etc. For example, physician servers 121, hospital servers 122, clinical trial servers 123, research lab servers 124, and/or laboratory information systems 125, etc., may each be connected to an electronic network 120, such as the Internet, through one or more computers, servers, and/or handheld mobile devices. According to an exemplary embodiment of the present disclosure, the electronic network 120 may also be connected to server systems 110, which may include processing devices 111. The tissue viewing platform 100 may also include a protocol tool 141 for determining protocols utilized by external users. In other examples, the protocol tool 141 may be operated separately from (e.g., by a different platform than) the tissue viewing platform 100.

The physician servers 121, hospital servers 122, clinical trial servers 123, research lab servers 124, and/or laboratory information systems 125 may create or otherwise obtain images of one or more patients' cytology specimen(s), histopathology specimen(s), slide(s) of the cytology specimen(s), digitized images of the slide(s) of the histopathology specimen(s), or any combination thereof. The physician servers 121, hospital servers 122, clinical trial servers 123, research lab servers 124, and/or laboratory information systems 125 may also obtain any combination of patient-specific information, such as age, medical history, cancer treatment history, family history, past biopsy or cytology information, etc. The physician servers 121, hospital servers 122, clinical trial servers 123, research lab servers 124, and/or laboratory information systems 125 may transmit digitized slide images and/or patient-specific information to server systems 110 over the electronic network 120. Server systems 110 may include one or more storage devices 109 for storing images and data received from at least one of the physician servers 121, hospital servers 122, clinical trial servers 123, research lab servers 124, and/or laboratory information systems 125. Server systems 110 may also include processing devices 111 for processing images and data stored in the one or more storage devices 109. Server systems 110 may further include one or more machine learning tool(s) or capabilities. For example, the processing devices 111 may include a machine learning tool for the tissue viewing platform 100, according to one embodiment. Alternatively or in addition, the present disclosure (or portions of the system and methods of the present disclosure) may be performed on a local processing device (e.g., a laptop).

The physician servers 121, hospital servers 122, clinical trial servers 123, research lab servers 124, and/or laboratory information systems 125 refer to systems used by pathologists for reviewing the images of the slides. In hospital settings, tissue type information may be stored in one of the laboratory information systems 125.

FIG. 1B illustrates an exemplary block diagram of a tissue viewing platform 100. For example, the tissue viewing platform 100 may include the slide analysis tool 101, a data ingestion tool 102, a slide intake tool 103, a slide scanner 104, a slide manager 105, a storage 106, a viewing application tool 108, and a protocol tool 141.

The slide analysis tool 101, as described in greater detail below, refers to a process and system for processing digital pathology slides (e.g., digitized images of slide-mounted histology or cytology specimens), and using one or more machine learning systems to analyze a given slide, according to an exemplary embodiment. The slide analysis tool 101 may determining measurements and/or diagnosis of digital medical images for one or more protocols. For example, the slide analysis tool 101 may determine a machine learning algorithm for each version of a particular protocol. These determined machine learning algorithms may be created and/or received through network 120.

The protocol tool 141, as described in detail below, refers to a process and system for processing digital pathology slides (e.g., digitalized images of a slide-mounted history or cytology specimens) and using machine learning or a rules based system for determining whether external designations match one or more of the protocols. An external designation may refer to metadata of a measurement and/or a diagnosis, and/or annotations of a digital medical image.

The data ingestion tool 102 refers to a process and system for facilitating a transfer of the digital pathology images to the various tools, modules, components, and devices that may be used for classifying and processing the digital pathology images, according to an exemplary embodiment.

The slide intake tool 103 refers to a process and system for scanning pathology slides and converting them into a digital form, according to an exemplary embodiment. The slides may be scanned with the slide scanner 104, and the slide manager 105 may process the images on the slides into digitized pathology images and store the digitized images in storage 106.

The viewing application tool 108 refers to a process and system for providing a user (e.g., a pathologist) with specimen property or image property information pertaining to digital pathology image(s), according to an exemplary embodiment. The information may be provided through various output interfaces (e.g., a screen, a monitor, a storage device, and/or a web browser, etc.).

The slide analysis tool 101, and one or more of its components, may transmit and/or receive digitized slide images and/or patient information and/or AI models (such as machine learning models) to server systems 110, physician servers 121, hospital servers 122, clinical trial servers 123, research lab servers 124, and/or laboratory information systems 125 over an electronic network 120. Further, server systems 110 may include one or more storage devices 109 for storing images, AI models, and data received from at least one of the slide analysis tool 101, the data ingestion tool 102, the slide intake tool 103, the slide scanner 104, the slide manager 105, and the viewing application tool 108. Server systems 110 may also include the processing devices 111 for processing images and data stored in the storage devices 109. Server systems 110 may further include one or more machine learning tool(s) or capabilities, e.g., due to the processing devices 111. Alternatively or in addition, the present disclosure (or portions of the system and methods of the present disclosure) may be performed on a local processing device (e.g., a laptop).

Any of the above devices, tools and modules may be located on a device that may be connected to an electronic network 120, such as the Internet or a cloud service provider, through one or more computers, servers, and/or handheld mobile devices.

FIG. 1C illustrates an exemplary block diagram of the slide analysis tool 101, according to an exemplary embodiment of the present disclosure. The slide analysis tool 101 may include a training image platform 131 and/or a target image platform 135.

The training image platform 131, according to one embodiment, may create or receive training images that are used to train a machine learning system to effectively analyze and classify digital pathology images. For example, the training images may be received from any one or any combination of the server systems 110, physician servers 121, hospital servers 122, clinical trial servers 123, research lab servers 124, and/or laboratory information systems 125. Images used for training may come from real sources (e.g., humans, animals, etc.) or may come from synthetic sources (e.g., graphics rendering engines, three-dimensional (3D) models, etc.). Examples of digital pathology images may include (a) digitized slides stained with a variety of stains, such as (but not limited to) hematoxylin and eosin (H&E), hematoxylin alone, immunohistochemistry (IHC), molecular pathology, etc.; and/or (b) digitized image samples from a 3D imaging device, such as micro computed tomography (micro-CT), CT, magnetic resonance imaging (MRI), etc.

The training image intake module 132 may create or receive a dataset comprising one or more training images corresponding to either or both of images of a human tissue and images that are graphically rendered. For example, the training images may be received from any one or any combination of the server systems 110, physician servers 121, and/or laboratory information systems 125. This dataset may be kept on a digital storage device. In some examples, the dataset may be comprised of a plurality of data subsets, where each data subset corresponds to a set of training images with annotations/markings of a different version of a protocol. The training slide characteristic module 133 may include one or more computing devices capable of, e.g., determining whether the training images have a sufficient level-of-quality for training a machine learning model. The training slide characteristic module 133 may further include one or more computing devices capable of, e.g., identifying whether a set of individual cells belong to a cell of interest or a background of a digitized image.

The target image platform 135 may include one or more computing devices capable of receiving a target dataset and applying a machine learning model to the received target dataset to determine a measurement and/or diagnosis for one or more protocols. In some examples, the target dataset may include one or more target images included in a case. Characteristics of the target dataset may include, but are not limited to, case complexity, case type, number of areas of interest per slide, amount of tissue per slide, and/or an image quality. For example, the target dataset may be received from the server systems 110, the physician servers 121, the hospital servers 122, the clinical trial servers 123, the research lab servers 124, and/or the laboratory information systems 125. The image intake module 136 may include one or more computing devices capable of, e.g., receiving a target dataset. Slide characteristic module determination 137 may include one or more computing devices capable of, e.g., applying a machine learning model to the target dataset to determine case complexity, case type, number of areas of interest, amount of tissue per slide and/or image quality. The slide characteristic module determination 137 may also be responsible for identifying the amount of image that one or more pathologist may need to review in connection with the inputted case. For instance, the slide characteristic module determination 137 may identify all prior cases that a patient has that may need to be reviewed/revisited.

The output interface 138 may include one or more computing devices capable of, e.g., outputting information about the measurements and/or diagnosis of a digital medical image (e.g., to a screen, monitor, storage device, web browser, etc.). In some examples, the output interface 138 may provide the various diagnosis and/or measurements to the protocol tool 141, e.g., to be used as an input for one or more other processes (e.g., information about the external designation and determined protocol utilized).

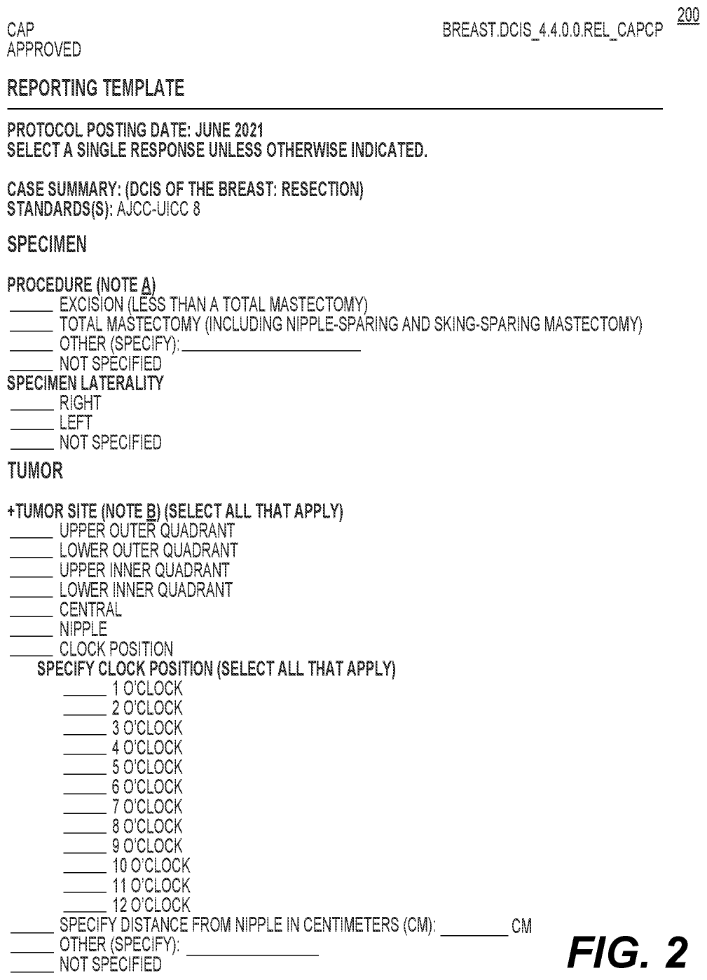

FIG. 2 depicts an exemplary section of a College of American Pathologist protocol. In particular, FIG. 2 depicts a section of the Protocol for the Examination of Resection Specimen from Patients with ductal Carcinoma in Situ (DCIS) 200 of the Breast version 4.4.0.0 released in June of 2021 (“Breast DCIS, Resection Protocol”). Additional versions of the Breast DCIS, Resection Protocol include, but are not limited to, Breast DCIS, Resection protocol were released in February 2020 (version 4.3.0.2), September 2019 (version 4.3.0.1), August 2019 (version 4.3.0.0), and February 2019 (version 4.2.0.0). The systems and methods described herein may be capable of receiving an external designation from a pathologist and determining a diagnosis and/or measurement for each of the protocol versions utilizing one or more machine learning systems. The systems and methods described herein may further compare and contrast the external designation to the determined diagnosis for each protocol and determine what protocol was utilized in the external designation.

The systems and methods described herein may be applied for all CAP protocols related to Breast (e.g., Breast DCIS, Resection; Breast DCIS, Biopsy; Breast Phyllodes Tumor; Breast Invasive, Resection; Breast Invasive, Biopsy; or Breast Biomarker Reporting), Central Nervous System, Endoctrine (e.g., Adrenal Gland, Appendix NET, Colon NET, Duodenum and Ampulla NET, Jejunum and Ileum NET, Pancreas Endocrine, Stomach NET, Thyroid, or Thyroid Biomarker Reporting), Gastrointestinal (e.g., Ampulla of Vater; Anus, Excision; Anus, Resection; Appendix; Colon and Rectum, Biopsy; Colon and Rectum, Resection; Colon and Rectum Biomarker Reporting; Distal Extrahepatic Bile Ducts; Esophagus; Gallbladder; GIST, Biopsy; GIST, Resection; GIST Biomarker Reporting; Hepatocellular Carcinoma; Intrahepatic Bile Ducts; Pancreas (Exocrine); Perihilar Bile Ducts; Small Intestine; Stomach; or Gastric HER2 Biomarker Reporting), Genitourinary (e.g., Kidney, Biopsy; Kidney, Resection; Penis; Prostate Needle Biopsy Case Level; Prostate Needle Biopsy

Specimen Level; Prostate, Resection; Prostate TURP; Testis Radical Orchiectomy; Testis Lymphadenectomy; Ureter, Renal Pelvis, Biopsy; Ureter, Renal Pelvis, Resection; Urethra, Biopsy; Urethra, Resection; Urinary Bladder, Resection; Urinary Bladder, or Biopsy), Gynecologic (e.g., Endometrium Uterus; Endometrium Biomarker Reporting *RETIRED*; Gynecologic, Biomarkers *NEW*; Ovary, Fallopian Tube, or Peritoneum; Trophoblastic Tumors; Uterine Cervix, Excision; Uterine Cervix, Resection; Uterine Sarcoma; Vagina, Biopsy; Vagina, Resection; or Vulva), Head and Neck (e.g., Larynx, Oral Cavity, Major Salivary Glands, Nasal Cavity and Paranasal Sinuses, Pharynx, Head and Neck Biomarker Reporting, or Head and Neck Biomarker Reporting), Hematologic (e.g., Bone Marrow, Hodgkin Lymphoma, Non-Hodgkin Lymphoma, or Plasma Cell Neoplasms), Ophthalmic (e.g., Retinoblastoma, or Uveal Melanoma), Pediatric (e.g., Ewing, Resection; Ewing, Biopsy; Germ Cell Tumor, Resection; Germ Cell Tumor, Biopsy; Hepatoblastoma, Resection; Hepatoblastoma, Biopsy; Neuroblastoma, Resection; Neuroblastoma, Biopsy; Rhabdomyosarcoma, Resection; Rhabdomyosarcoma, Biopsy; Wilms, Resection; or Wilms, Biopsy), Skin (e.g., Skin, Melanoma, Biopsy; Skin, Melanoma, Excision; Melanoma Biomarker Reporting; or Merkel Cell Carcinoma), Thorax (e.g., Lung, Resection; Lung Biomarker Reporting; Pleural Mesothelioma; or Thymus), Bone and Soft Tissue (e.g., Bone, Biopsy; Bone, Resection; Soft Tissue, Biopsy; or Soft Tissue, Resection), or General (e.g., DNA Mismatch Repair Biomarker Reporting; Generic Template, Biopsy; Generic Template, Resection; or General IHC Quantitative Biomarkers). The systems and methods described herein may also be utilized on hospital specific guidelines and protocols.

The system and techniques described herein may be utilized to help pathologists/researchers stay up to date with the most recent protocol, synoptic reports, and/or worksheets. The system may determine one or more machine learning or artificial intelligence (AI) systems that are trained on each existing and/or new protocol, synoptic report, and/or worksheet. The system may be utilized to compare measurements and/or the diagnosis from a pathologist/user with the one or more machine learning systems to determine whether the latest guidelines are being used by the pathologist/user. If the latest guidelines are not being used, the system may notify the pathologist/user that they are not utilizing the newest guidelines. The latest version of the guidelines may be provided, and/or automatically incorporated into the system.

Exemplary information on the Breast DCIS, Resection Protocol includes procedure, specimen laterality, tumor site, specific clock positions, etc.

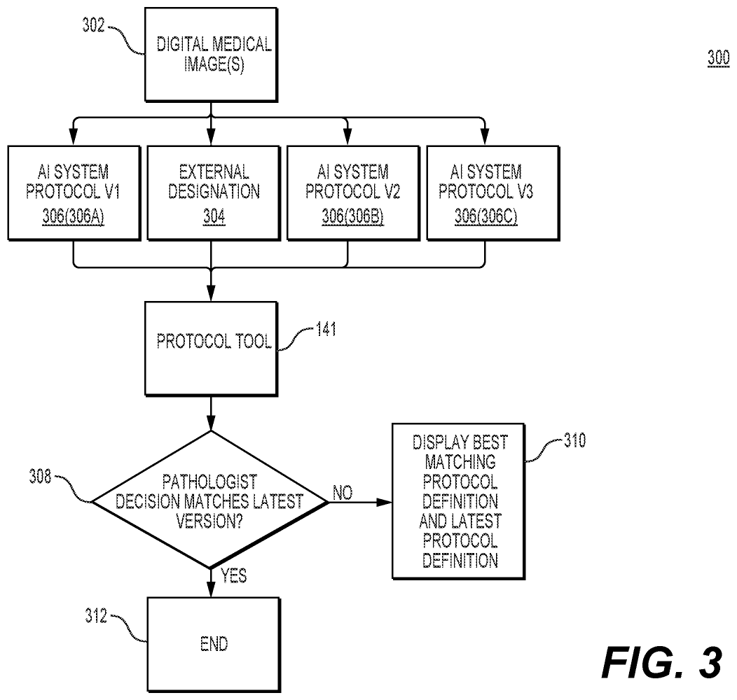

FIG. 3 depicts an exemplary block diagram 300 illustrating a process for using a trained system to determine an external designation protocol. The trained system may be implemented by the protocol tool 141 of the tissue viewing platform 100. The protocol tool 141 may utilize the trained system to determine the protocol version utilized by a particular external designation 304 of a received digital medical image 302. In one example, this may be done by comparing the external designation 304 to one or more machine learning systems 306 trained on different versions of the protocol. The protocol tool 141 may output 308 a determination of what protocol version that matches the external designation (e.g., a similarity match). Further, the trained system may display and/or output that no protocol matches the external designation. If the output 308 does not have the external designation matching the newest and/or preferred protocol, the trained system may output 310 the protocol utilized in the external designation and the newest and/or preferred protocol to a user. If the output 308 matches the newest and/or preferred protocol, the trained system may record that the correct protocol was utilized and end the analysis 312.

The protocol tool 141, as well as the determined machine learning systems 306 may receive the user performing an external designation 304 and the one or more digital medical images 302 to be analyzed (e.g., for a measurement or diagnosis). The digital medical images may include (a) digitized slides stained with a variety of stains, such as (but not limited to) H&E, Hematoxylin alone, IHC, molecular pathology, etc.; and/or (b) digitized image samples from a 3D imaging device, such as micro-CT. The digital medical images may also be referred to as whole slide images (WSIs). In some examples, other image related metadata may be imported from the storage devices 109, clinical trial server 123, physician servers 121, laboratory information system 125, research lab servers 124, hospital servers 122, or from an external network. This metadata may include the tissue type and the general protocol (e.g., the type of protocol to be applied). Further, these digital medical images 302 may be imported through external storage devices such as a flash drive. The protocol tool 141 may be capable of constantly receiving new digital medical images 302 and determining an external designation 304 and determined machine learning systems 306 that correspond to the newly received digital medical images 302.

The protocol tool 141 may further receive an external designation 304 corresponding to a received digital medical images 302. The external designation 304 may be metadata of a measurement and/or diagnosis of the received digital medical images 302. The external designation 304 may further include metadata as to what protocol or guideline is utilized. An exemplary external designation may be a measurement of the length of cancer for a particular medical digital image of a tissue with breast cancer.

The protocol tool 141 may further determine one or more machine learning systems 306 trained per each version of a protocol for a specific guideline. In another example, the protocol tool 141 may determine a machine learning system 306 trained on the three most recent version of a protocol. For instance, if protocol tool 141 receives one or more external designations 304 and one or more digital medical images 302 that are for a Breast DCIS, biopsy, etc., the protocol tool 141 may then determine one or more machine learning systems 306 for one or more of the protocols corresponding to the specific guideline. In one example, the protocol tool 141 may receive, e.g., via server systems 110, the physician servers 121, the hospital servers 122, the clinical trial servers 123, the research lab servers 124, and/or the laboratory information systems 125 the determined machine learning systems 306 for each protocol version, once the protocol tool 141 is determined. In another example, the protocol tool 141 may determine the machine learning systems 306 by training the machine learning systems. The protocol tool 141 may utilize the systems, methods, and techniques of U.S. Provisional Patent Application No. 63/341,504 to determine the machine learning systems 306. The system may be trained to automatically look for new guidelines and/or initiate training or retraining the machine learning model(s).

In one example, the protocol tool 141, may determine a machine learning system 306 for each version of a particular protocol. In another example, the protocol tool 141 may determine a predetermined number of machine learning systems for the latest versions of the protocols (e.g., the protocol tool 141 may determine three systems such as machine learning system 306A, machine learning system 306B, and machine learning system 306C).

Each of the machine learning systems 306 may be applied to the one or more received digital medical images 302 to determine a measurement and/or diagnosis. This determined measurement and/or diagnosis may then be outputted to the protocol tool 141. The protocol tool 141 may then compare the external designation 304 to the measurement and/or diagnosis of the machine learning systems 306. This may be performed for each individual digital medical image 302 received. In one example, a rule-based logic algorithm may be utilized to compare the measurement and/or diagnosis of the external designation 304 to the machine learning systems 306. The algorithm may compare the closest measurement (e.g., length of cancer) and determine which measurement of the machine learning systems 306 is closest to that of the external designation 304. This step may be referred to as an output 308 (e.g., the similarity match). In another example of a output 308 (e.g., a similarity match), when comparing a diagnosis (e.g., comparing cancer grading), the protocol tool 141 may utilize a trained system to determines what machine learning system 306 output (and the machine learning system's 306 protocol version) is closest to the external designation. The protocol tool 141 may assess the similarities and differences between these outputs using machine learning techniques.

The trained system of the protocol tool 141 may be a trained machine learning (ML) system. Exemplary types of ML systems that may be trained and implemented may include, but are not limited to, reactive machines and limited memory machines. In some examples, the ML system may include a neural network for objective function approximation. The ML systems training may be based on the inputs received. During training, the ML system may first automatically determine a type of input, for example the input may be a measurement length. The ML system may then perform a regression based method with mean squared error loss, for example. The ML system may then perform categorical regression and binary regression and compare the results. The system may then automatically select the loss function based on the comparison.

If the output 308 (e.g., similarity match) outputted from protocol tool 141 determines that the external designation 304 matches a machine learning systems 306 that corresponds to the newest and/or preferred version of a protocol, the protocol tool 141 may end analysis 312. Prior to ending analysis 312, the system may record that the external designation matches the newest and/or preferred protocol or guideline. Further, the system may output via the server systems 110, the physician servers 121, the hospital servers 122, the clinical trial servers 123, the research lab servers 124, and/or the laboratory information systems 125 that the newest and/or preferred protocol or guideline was utilized. The newest guideline may be displayed and/or outputted to an external user (e.g., through the viewing application tool 108).

If the output 308 (e.g., similarity match), outputted from the protocol tool 141, determines that external designation 304 does not match the newest and/or preferred protocol, the protocol tool 141 may output and/or display a notification 310 that the newest and/or preferred guidelines version is not being utilized. The output and/or display notification 310 may include what version of the guideline was utilized in the measurement and/or diagnosis, and what version of the guidelines is newest and/or preferred. The protocol tool 141 may further alert the user, hospital, superior of the individual, or other predetermined party as to what protocol was utilized. For example, if an individual used a previous version of a guideline within their external designations 304 a predetermined number of times, the protocol tool 141 may be programmed to send an alert to the individual's superior. Alternatively, the protocol tool 141 may provide training for a new guideline if a user deviates from the newest and/or preferred guideline a predetermined number of times and/or a predetermined portion of uses. The training may be related to a lab/hospital's quality policy such as the International Organization for Standardization's (ISO) quality management system. In another example, the protocol tool 141 may be utilized as a statistical measure of what guidelines particulars pathologist use for their work. In this example, the system may track what guidelines are employed by each user for each type of case.

In one example, the protocol tool 141 may determines that external designation 304 associated with a slide are not compatible (e.g., the similarity match is negative) with the latest guideline. In response to this determining, the protocol tool 141 may automatically make and/or suggest an update to the annotations, evaluations, and/or metadata to bring them into accordance with the latest version of the guidelines. For example, the annotations may identify regions that should and/or should not have been annotated. The annotations may further provide the ground and/or terms on which the suggestions are based with respect to the particular protocol. The evaluations may identify subjects whose evaluation should be rendered differently under the new protocol. For example, if a digital medical image 302 displays a gap between two cancerous regions of 2 millimeters, the protocol tool 141 may automatically determine using a machine learning systems 306 of the preferred version, that the two regions should be measured as one region (e.g., if the machine learning system 306 of the newest and/or preferred protocol). If the external designation 304 (e.g., initial analysis) by the user/pathologist measured two regions rather than one, the protocol tool 141 may automatically determine that the slide analysis is out of compliance with the latest guidelines. The protocol tool 141 might automatically update any external designation (e.g., a annotations, evaluations, and/or metadata) to indicate that the cancer is one larger region (e.g., update the external designation). The user/pathologist might be informed of automatic updates to annotations, evaluations, and/or metadata that have been applies by the system.

In another example, the similarity match may determine that the external designation 304 does not match any of the outputs from the determined machine learning systems 306. In this example, the system may output the newest and/or preferred guideline to a user and notify a user that no version of the guideline and/or protocol matches the external designation.

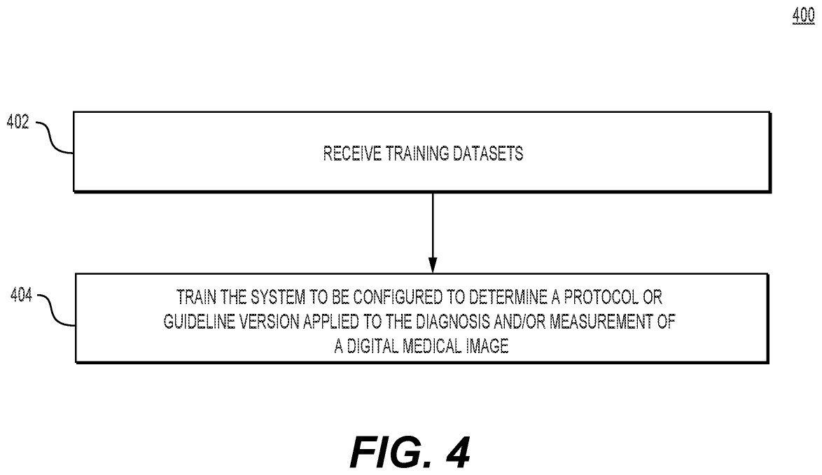

FIG. 4 is a flowchart of an example method for training a system to determine an external designation protocol, according to techniques presented herein. The method 400 of FIG. 4 depicts steps that may be performed by, for example, the protocol tool 141. Alternatively, the method 400 may be performed by an external system, where the trained system may be provided to the protocol tool 141 for implementation.

At step 402, a plurality of training datasets may be received. Training datasets may include digital medical images and corresponding metadata with diagnosis and/or measurements. Examples of digital medical images may include (a) digitized slides stained with a variety of stains, such as (but not limited to) H&E, hematoxylin alone, IHC, molecular pathology, etc., and/or (b) digitized tissue samples from a 3D imaging device, such as micro-CT. Further, the metadata may include information as to what protocol/guideline and what version of the protocol/guideline has been applied to the diagnosis and/or measurement. In one example, the metadata may include a diagnosis and/or measurement for each version of a particular protocol or guideline for the corresponding digital medical image.

At step 404, the system may be trained using one or more of the datasets. For example, the system may use the received datasets to train the system to be configured to determine what version of a protocol or guideline has been utilized for an external designation (e.g., a received measurement and/or diagnosis). As previously discussed in reference to FIG. 3, the trained system may be a trained machine learning system or may apply rule-based logic depending on the optimization.

The system may be trained as a multi-class classification problem where the possible versions of a protocol may have been used in different training datasets from step 402. To account for the protocols that was not considered and/or was recently added during training, a nominal class labeled “unsure” may be created for the model to express a confidence regarding its outputted answer. In training the system, may be developed so as the model (e.g., the protocol tool 141) has two components where the first component is responsible for learning good representations/features to distinguish one version of a protocol from another in a latent space. The other component may use the features/representations to output a specific classification. The train datasets of 402 may be created based on the scan and case year information associated with each groups of digital medical images.

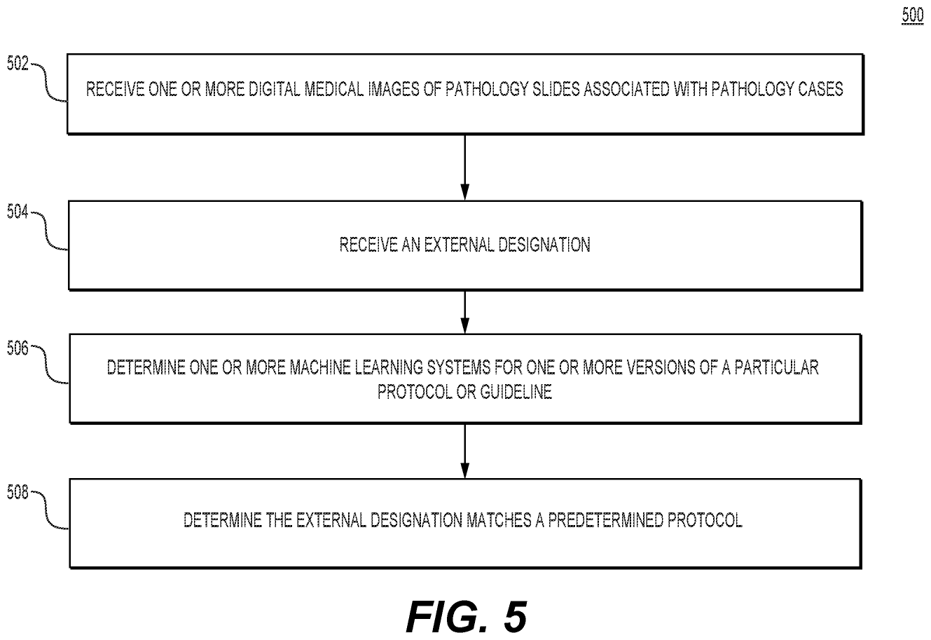

FIG. 5 is a flowchart illustrating an example method 500 for using a trained system to determine an external designation protocol, according to one or more exemplary embodiments herein. The exemplary method 500 (e.g., steps 502-508) of FIG. 5 depicts steps that may be performed by, for example, the protocol tool 141. These steps may be performed automatically or in response to a request from a user (e.g., a pathologist, a department or laboratory manager, an administrator, etc.). Alternatively, the method 500 may be performed by any computer process system capable of receiving image inputs such as device 700 and capable of storing and executing the trained system described in FIG. 4.

At step 502, the trained system may receive one or more digital medical images of pathology slides associated with pathology cases. The digital medical images may include (a) digitized slides stained with a variety of stains, such as (but not limited to) H&E, Hematoxylin alone, IHC, molecular pathology, etc.; and/or (b) digitized image samples from a 3D imaging device, such as micro-CT. The digital medical images may also be referred to as whole slide images (WSIs).

At step 504, the trained system may receive an external designation corresponding to the one or more digital medical images. The external designation may be metadata of a measurement and/or diagnosis of the digital medical image. The trained system may receive an external designation for each received digital medical image.

At step 506, the trained system may determine one or more machine learning systems for one or more versions of a particular protocol or guideline. The particular protocol or guideline may correspond to the protocol or guideline used in the external designation from step 504. In one example, the trained system may determine a set number of machine learning systems. For example, the trained system may determine a machine learning system for the three most recent versions of a particular guideline or protocol. In another example, the trained system may determine a machine learning system for all versions of a particular guideline or protocol. The newest and/or preferred guideline or protocol may be referred to as predetermined guideline. The determine machine learning systems from step 506 may be applied to the received one or more digital medical images from step 502. The outputs, as well as the external designation may be fed into the trained system.

At step 508, the trained system may determine whether the external designation matches a predetermined protocol. This may be done by comparing the external designation to the outputs of the determined machine learning systems and performing a similarity match to determine which version of a protocol or guideline was utilized in the external designation. For example, if the external designation has a most similar measurement and/or diagnosis to the output of the determined machine learning system trained on the most recent and/or preferred protocol or guideline, the system may end analysis on that particular digital medical image. The trained system may further record and/or output that the proper protocol or guideline was utilized within the external designation. If the similarity match performed by the trained system determine the preferred and/or newest protocol or guideline is not used, the trained system may output and/or display a notification that the newest and/or preferred guidelines version is not being utilized. The newest and/or preferred guideline may be displayed to a user. Further, the trained system may record that an older version of the protocol or guideline is associated with the external designation.

In one exemplary use of the systems and methods described herein, the trained system may be utilized by hospitals or research centers to track what guidelines are utilized by their employees. This may allow for hospitals to have a greater understanding of what guidelines are currently being used and how long it takes for guidelines to be utilized by individuals and the hospital as a whole. The system may accomplish this by tracking and recording what guidelines are utilized for each analysis performed on a digital medical image. .

For example, the trained system may allow for a notification as to when a predetermined percentage of workers have begun using an updated guideline. As an example, the system may be programmed to notify the hospital when over 75% of pathologists have begun utilizing a new/updated guideline.

For example, the system may first receive a digital medical image. Next, a pathologist may perform a diagnosis and/or measurement on the inputted digital medical image. The trained system may receive the inputted diagnosis and/or measurement. The trained system may then run machine learning/AI system protocols for each protocol or guideline version of the diagnosis/measurement performed. Next, the trained system may compare the diagnosis/measurements of the pathologist with the diagnosis/measurement of the machine learning/AI system(s). Specifically, the trained system may determine which version of the guidelines was utilized by the pathologist. If the pathologist used the latest (or some other preferred) version of the guidelines, the system may end analysis of the WSI and record that the pathologist used the latest (or otherwise preferred) guidelines. If the system matches the diagnosis/measurement with a previous version (or non-preferred version) of the guidelines, the system may notify the pathologist/user that they are using an outdated (or otherwise non-preferred) guideline and further provide the latest (preferred) guidelines to the user. The system may then record, for each use, what version of the guidelines was utilized. The system may track and display the statistics as to which guidelines are being used by all users.

In one exemplary use of the systems and methods described herein, the trained system may be utilized to help pathologist create new tools. The trained system may record and study the different guidelines utilized by a plurality of pathologists/users of the system. The trained system may then develop and refine guidelines based on the pathologist preference of certain guidelines or modifications to certain guidelines.

For example, the trained system may recognize that a majority of hospital workers perform a measurement of cancer in a way that deviates from a new guideline, but does not match any older versions of a guideline. If a majority of users deviate from a new (or any known) guideline in a similar way, the system may recognize this and notify the hospital/research center that a guideline may need to be updated or created to reflect how users have performed measurements.

For example, the trained system may first receive a digital medical image. Next, a pathologist/user may perform a diagnosis or measurement on the inputted digital medical image. The trained system may receive the inputted diagnosis or measurement. The trained system may then run machine learning and/or AI system protocols for a plurality of versions of the diagnosis/measurement performed. Next, the system may compare the diagnosis/measurements of the pathologist with the diagnosis/measurement of the machine learning/AI Systems. Specifically, the system may determine which version of the guidelines was utilized by the pathologist. If the pathologist used the latest version of the guidelines, the system may end analysis of the digital medical image and record that the pathologist used the latest guidelines. If the system matches the diagnosis/measurement with a previous version of the guidelines, the system may notify the pathologist that they are using an outdated guideline and further provide the latest guidelines to the user. The system may then record, for each use, what version of the guidelines was utilized. The system may record if user analysis does not match any version of a guideline. The system may suggest a new guideline to a hospital/research center if a certain percentage of users deviate from a guideline in a similar manner.

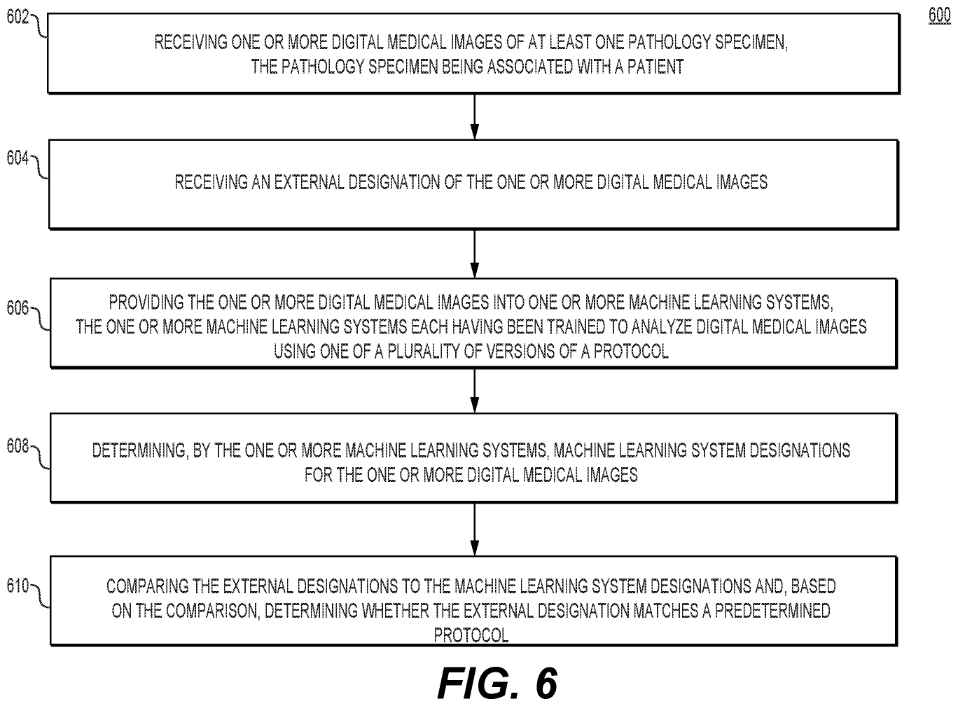

FIG. 6 is a flowchart illustrating an example method 600 for determining an external designation's protocol. At step 602, one or more digital medical images of at least one pathology specimen may be received, the pathology specimen being associated with a patient.

At step 604, an external designation of the one or more digital medical images may be received.

At step 606, the one or more digital medical images may be provided to one or more machine learning systems, the one or more machine learning systems each having been trained to analyze medical images using one of a plurality of versions of a protocol.

At step 608, machine learning system designations for the one or more digital medical images may be determined by the one or more machine learning systems.

AT step 610, the external designation may be compared to the machine learning system designations and, based on the comparison, determining whether the external designation matches a predetermined protocol.

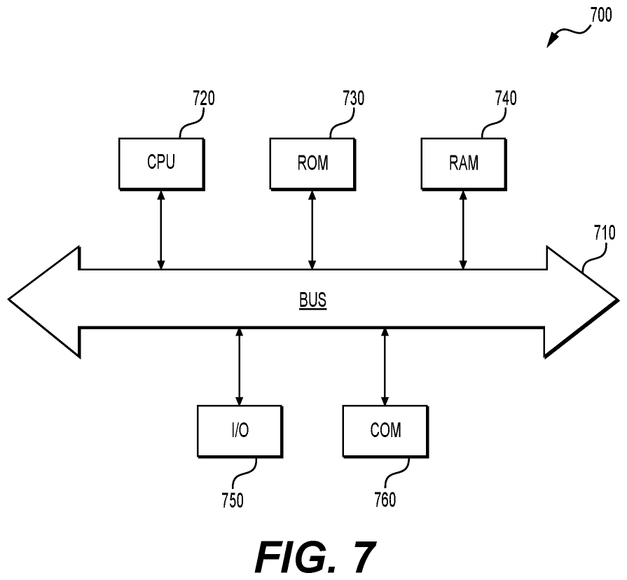

As shown in FIG. 7, device 700 may include a central processing unit (CPU) 720. CPU 720 may be any type of processor device including, for example, any type of special purpose or a general-purpose microprocessor device. As will be appreciated by persons skilled in the relevant art, CPU 720 also may be a single processor in a multi-core/multiprocessor system, such system operating alone, or in a cluster of computing devices operating in a cluster or server farm. CPU 720 may be connected to a data communication infrastructure 710, for example a bus, message queue, network, or multi-core message-passing scheme.

Device 700 may also include a main memory 740, for example, random access memory (RAM), and also may include a secondary memory 730. Secondary memory 730, for example a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 730 may include similar means for allowing computer programs or other instructions to be loaded into device 700. Examples of such means may include a program cartridge and cartridge interface (such as that found in video game devices), a removable memory chip (such as an EPROM or PROM) and associated socket, and other removable storage units and interfaces, which allow software and data to be transferred from a removable storage unit to device 700.

Device 700 also may include a communications interface (COM) 760. Communications interface 760 allows software and data to be transferred between device 700 and external devices. Communications interface 760 may include a modem, a network interface (such as an Ethernet card), a communications port, a PCMCIA slot and card, or the like. Software and data transferred via communications interface 760 may be in the form of signals, which may be electronic, electromagnetic, optical or other signals capable of being received by communications interface 760. These signals may be provided to communications interface 760 via a communications path of device 700, which may be implemented using, for example, wire or cable, fiber optics, a phone line, a cellular phone link, an RF link or other communications channels.

The hardware elements, operating systems, and programming languages of such equipment are conventional in nature, and it is presumed that those skilled in the art are adequately familiar therewith. Device 700 may also include input and output ports 750 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Throughout this disclosure, references to components or modules generally refer to items that logically may be grouped together to perform a function or group of related functions. Like reference numerals are generally intended to refer to the same or similar components. Components and/or modules may be implemented in software, hardware, or a combination of software and/or hardware.

The tools, modules, and/or functions described above may be performed by one or more processors. “Storage” type media may include any or all of the tangible memory of the computers, processors or the like, or associated modules thereof, such as various semiconductor memories, tape drives, disk drives and the like, which may provide non-transitory storage at any time for software programming.

Software may be communicated through the Internet, a cloud service provider, or other telecommunication networks. For example, communications may enable loading software from one computer or processor into another. As used herein, unless restricted to non-transitory, tangible “storage” media, terms such as computer or machine “readable medium” refer to any medium that participates in providing instructions to a processor for execution.

The foregoing general description is exemplary and explanatory only, and not restrictive of the disclosure. Other embodiments may be apparent to those skilled in the art from consideration of the specification and practice of the invention disclosed herein. It is intended that the specification and examples be considered as exemplary only.

What is claimed is:

1. A computer-implemented method for processing electronic medical images comprising:
receiving one or more digital medical images of at least one pathology specimen, the pathology specimen being associated with a patient;
receiving an external designation of the one or more digital medical images;
providing the one or more digital medical images to one or more machine learning systems, the one or more machine learning systems each having been trained to analyze medical images using one of a plurality of versions of a protocol;
determining, by the one or more machine learning systems, machine learning system designations for the one or more digital medical images; and
comparing the external designation to the machine learning system designations and, based on the comparison, determining whether the external designation matches a predetermined protocol.

2. The method of claim 1, wherein the predetermined protocol corresponds to a most recently determined protocol.

3. The method of claim 1, further including:
outputting to one or more users that the predetermined protocol was used in the external designation.

4. The method of claim 1, further including:
outputting whether the external designation does not match any of the machine learning system designations.

5. The method of claim 1, further including:
upon determining the external designation does not match the predetermined protocol, outputting the predetermined protocol to a user or external system.

6. The method of claim 1, further including:
outputting the protocol of the machine learning designation that most closely matches the external designation.

7. The method of claim 1, further including:
determining a new protocol has been developed;
updating the predetermined protocol to be the new protocol; and
training a new machine learning system based on the new protocol.

8. A system for processing electronic medical images, the system comprising:
at least one memory storing instructions; and
at least one processor configured to execute the instructions to perform operations comprising:
receiving one or more digital medical images of at least one pathology specimen, the pathology specimen being associated with a patient;
receiving an external designation of the one or more digital medical images;
providing the one or more digital medical images to one or more machine learning systems, the one or more machine learning systems each having been trained to analyze medical images using one of a plurality of versions of a protocol;
determining, by the one or more machine learning systems, machine learning system designations for the one or more digital medical images; and
comparing the external designation to the machine learning system designations and, based on the comparison, determining whether the external designation matches a predetermined protocol.

9. The system of claim 8, wherein the predetermined protocol corresponds to a most recently determined protocol.

10. The system of claim 8, further including:
outputting to one or more users that the predetermined protocol was used in the external designation.

11. The system of claim 8, further including:
outputting whether the external designation does not match any of the machine learning system designations.

12. The system of claim 8, further including:
upon determining the external designation does not match the predetermined protocol, outputting the predetermined protocol to a user or external system.

13. The system of claim 8, further including:
outputting the protocol of the machine learning designation that most closely matches the external designation.

14. The system of claim 8, further including:
determining a new protocol has been developed;
updating the predetermined protocol to be the new protocol; and
training a new machine learning system based on the new protocol.

15. A non-transitory computer-readable medium storing instructions that, when executed by a processor, perform operations processing electronic medical images, the operations comprising:
receiving one or more digital medical images of at least one pathology specimen, the pathology specimen being associated with a patient;
receiving an external designation of the one or more digital medical images;
providing the one or more digital medical images to one or more machine learning systems, the one or more machine learning systems each having been trained to analyze medical images using one of a plurality of versions of a protocol;
determining, by the one or more machine learning systems, machine learning system designations for the one or more digital medical images; and
comparing the external designation to the machine learning system designations and, based on the comparison, determining whether the external designation matches a predetermined protocol.

16. The computer-readable medium of claim 15, wherein the predetermined protocol corresponds to a most recently determined protocol.

17. The computer-readable medium of claim 15, further including:
outputting to one or more users that the predetermined protocol was used in the external designation.

18. The computer-readable medium of claim 15, further including:
outputting whether the external designation does not match any of the machine learning system designations.

19. The computer-readable medium of claim 15, further including:
upon determining the external designation does not match the predetermined protocol, outputting the predetermined protocol to a user or external system.

20. The computer-readable medium of claim 15, further including:
outputting the protocol of the machine learning designation that most closely matches the external designation.
